(19) United States
(12) Patent Application Publication
(10) Pub. No.: US 2006/0264488 A1
(43) Pub. Date: Nov. 23, 2006

(54) TUBULAR PROTEINURIA AS AN INDICATOR FOR ELEVATED CARDIOVASCULAR RISK

(75) Inventor: Joachim SCHRADER, Cloppenburg (DE)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 11/420,092

(22) Filed: May 24, 2006

Related U.S. Application Data
(63) Continuation of application No. PCT/EP04/13511, filed on Nov. 26, 2004.

(30) Foreign Application Priority Data
Nov. 26, 2003 (EP) 03027312.2

(51) Int. Cl. A61K 31/4184; A61K 31/401

(52) U.S. Cl. 514/381; 514/423

(57) ABSTRACT

The invention relates to the use of an ACE inhibitor or an angiotensin receptor antagonist for the preparation of a pharmaceutical composition for reducing tubular proteinuria oral/or α₁-microglubulin in a non-diabetic human individual for reducing the risk of a cardiovascular event.

Coronary heart disease is still the leading cause of morbidity and mortality in the United States. Risk factors for cardiovascular disease may be directly causative, may be secondary manifestations of a more basic underlying metabolic abnormality or may represent early symptoms of the disease. Several risk factors are associated with an increased incidence of cardiovascular disease, including cigarette smoking, hypertension, dyslipidemia, diabetes mellitus, microalbuminuria, obesity, a sedentary lifestyle, gender and poor nutrition. The effects of risk factors in adults are additive: the greater the number of high-risk factors present, the greater the risk of cardiovascular disease. Intervention of some of the reversible, directly causative risk factors has been effectively used to lower the risk of developing cardiovascular disease, and the incidence of cardiovascular events such as stroke or myocardial infarction.

It is known that microalbuminuria, which is a form of glomerular proteinuria is an indicator of cardiovascular risk factors and cardiovascular morbidity.

It has now unexpectedly been found that tubular proteinuria and elevated urinary levels of α₁-microglobulin are independently indicative of an increased risk of developing cardiovascular disease and cardiovascular mortality.

It has moreover been found that by the reduction of tubular proteinuria and/or urinary α₁-microglobulin lowers the incidence of cardiovascular events.

It has thus been shown that modulators of the angiotensin/renin-system, such as the angiotensin converting enzyme (ACE) inhibitors or angiotensin receptor antagonists may effectively be used in lowering tubular proteinurea and α₁-microglobulin.

Moreover, it has been established in a large scale interventive trial that such therapeutic intervention in individuals exhibiting tubular proteinuria or elevated urinary a₁-miroglobulin will effectively reduce the risk of cardiovascular events.

In accordance with the above findings the present invention provides a new screening method for the assessment of the cardiovascular risk of an individual.

Moreover the screening method for the assessment of the cardiovascular risk according to the present invention is easier, faster and more reliable than the screening method using microalbuminuria as a predictor of cardiovascular events, as the determination of tubular proteinuria and α₁-mircroglobulin may be determined by standard procedures.

In a further aspect the present invention also provides for a pharmaceutical composition comprising a modulator of the angiotensin/renin-system, such as an angiotensin converting enzyme (ACE) inhibitor or an angiotensin receptor antagonists for reducing tubular proteinuria and/or urinary α-microglobulin in non-diabetic patients for reducing the risk of a cardiovascular event.

Therapeutic intervention following diagnosis with the diagnostic screening method of the invention may be carried out on asymptomatic healthy individuals as well as on patients with hypertension or manifest cardiovascular disease.

Preferred ACE-inhibitors according to the present invention are ramipril, perindopril, trandolapril, lisinopril, enalapril or captopril and their pharmaceutically acceptable salts and derivatives.

As angiotensin antagonists for use according to the present invention lorsartan, candesartan, valsartan, irbesartan, olmesartan, eprosartan or telmisartan their pharmaceutically acceptable salts and derivatives are preferred.

Urinary analysis may be carried out by any standard laboratory test. The determination of urinary albumin is preferably carried out by quantitative protein measurement (nephelometry) and SDS-electrophoresis.

Commercially test kits are available, such as the turbidimetric test, Tinaquant alpha-microglobulin by Roche Diagnostics.

In tubular proteinuria, small molecular weight proteins may be found including retinol-binding protein, α₁-microglobulin, β-₂-microglobulin lysozyme, light chains, haemoglobin and myoglobin.

1. Use of an ACE inhibitor or an angiotensin receptor antagonist for the preparation of a pharmaceutical composition for reducing tubular proteinuria and/or α₁-microglobulin in a non-diabetic human individual for reducing the risk of a cardiovascular event.

2. The use according to claim 1, wherein the ACE inhibitor is selected from ramipril, perinolopril, trandolapril, lisinopril, enalapril or captopril.

3. The use according to claim 1, wherein the angiotensin antagonist is lorsartan, candesartan, valsartan, irbesartan, olmesartan, eprosartan and telmisartan.

4. The use according to claim 1, wherein the cardiovascular event is selected from stroke or myocardial infarction.

5. An in vitro diagnostic method for the assessment of the risk of a human individual to suffer from a cardiovascular disease or a cardiovascular event comprising determining the level of urinary β₂-microglobulin or other tubular proteins and α₁-microglobulin.
